(19) United States
(12) Patent Application Publication
(10) Pub. No.: US 2024/0035921 A1
(43) Pub. Date: Feb. 1, 2024

(54) DETERMINING A CONDITION OF A COMPOSITE COMPONENT

(71) Applicant: HOCHSCHULE TRIER, Trier (DE)

(72) Inventors: Philipp LENZ, Konz (DE); Klaus-Peter KOCH, Wadern (DE); Martin QUINTUS, Simmern (DE); Christoph MAIER, Aach (DE)

(73) Assignee: HOCHSCHULE TRIER, Trier (DE)

(21) Appl. No.: 18/226,425

(22) Filed: Jul. 26, 2023

(30) Foreign Application Priority Data
Jul. 28, 2022 (DE) 10 2022 118 916.3

(51) Int. Cl. G01M 5/00

(52) U.S. Cl. CPC G01M 5/0091; G01M 5/0033; G01M 5/0066

(57) ABSTRACT

A device for determining a condition of a composite component, including: an input interface for receiving measurement data with information on a condition of the composite component; an analysis unit for determining a condition of the composite component based on the measurement data; and an output interface for transmitting the determined condition; wherein the measurement data includes an electrical charge transfer and/or electrical voltage, which can be generated by the composite component through mechanical excitation of the composite component; and the analysis unit is designed and configured to determine the condition of the composite component on the basis of the electrical charge separation and/or electrical voltage. The present invention further relates to a corresponding system and method.

The present invention relates to a device for determining a condition of a composite component and a corresponding system and method.

Determining of a condition of composite materials is required in many applications. In particular, the internal structure, changes in the individual materials and material composites are of special interest for the stability and mechanical properties of the composite materials.

For this purpose, it is known to estimate wear of composite materials based on model calculations and estimated load histories.

Furthermore, it is known to determine wear, current load or aging effects with sensors attached to the exterior of the composite materials and/or measurements performed from the outside.

However, the aforementioned possibilities include approximations and/or point measurements. A simple and/or direct determination of the composite material condition, the load history and/or the current load of the composite material is sometimes not easily possible.

Therefore, there is a need to improve the measurement of a condition of composite materials.

For this reason, the present invention confronts the task of specifying a possibility for an optimized determination of a condition of composite materials. In particular, an instantaneous mechanical load, a load history, aging and/or defects are to be detectable.

The above problem is solved by a device for determining a condition of a composite component, comprising:
an input interface for receiving measurement data with information on a condition of the composite component;
an optional input interface for detecting and/or generating a mechanical excitation of the composite component;
an analysis unit for determining a condition of the composite component based on the measurement data and optionally the mechanical excitation; and
an output interface for transmitting the determined condition; wherein
the input interface may preferably be suitable to detect electrical charge transfers and/or electrical voltages on the composite material and/or to receive sensor data from a sensor;
the mechanical excitation can be provided by the device or through the usual use of the composite component;
the measurement data comprises an electrical charge transfer and/or electrical voltage, which can be generated by the composite component through mechanical excitation of the composite component; and
the analysis unit is designed and configured to determine the condition of the composite component on the basis of the electrical charge separation and/or electrical voltage.

Furthermore, the above problem is solved by a system for determining a condition of a composite component, comprising:
a device as previously defined; and
an excitation unit for exerting on the composite component and generating a predefined mechanical excitation of the composite component.

Finally, the above problem is solved by a method for determining a condition of a composite component comprising the steps of:
receiving measurement data with information on a condition of the composite component;
determining a condition of the composite component based on the measurement data; and
transmitting the determined condition; wherein
the measurement data comprises an electrical charge separation and/or electrical voltage generated by the composite component through mechanical excitation of the composite component; and
determining a condition of the composite component comprises determining the condition of the composite component based on the electrical charge separation and/or electrical voltage.

In particular, a measurement and a reception of the excitation of the composite component take place.

Preferred embodiments of the invention are described in the dependent claims. It is to be understood that the features mentioned above and those to be explained below can be used not only in the combination indicated in each case, but also in other combinations or on a stand-alone basis, without departing from the scope of the present invention. In particular, the method may be implemented according to the embodiments described for the device and/or the system in the dependent claims.

It is to be understood that the method can also be implemented in the form of a computer program.

By means of a device comprising an input interface, an analysis unit and an output interface, a simple and cost-efficient device can be created which, in particular, can be produced in large quantities and is suitable for mobile applications. In doing so, the device makes use of current research results, in particular from Trier University of Applied Sciences. It was found that mechanical loads on conductive and non-conductive material composites cause electrical charge transfers. The magnitude of the electrical charge separation or electrical charge transfer thus generated is related to an experienced, in particular mechanical, load on the material composites. These electrical charges or electrical voltages can be measured and allow direct conclusions to be drawn on the condition of the material composites. This finding is used in the analysis unit, which is designed and configured to determine the condition of the composite component on the basis of the electrical charge separation and/or electrical voltage. In particular, models, empirical data and/or neural networks can be used here to analyze the data. It is to be understood that the composite component preferably comprises at least one electrically conductive material. It is further to be understood that a suchlike composite component may be created by attaching a conductive material to an outer surface of any component. In particular, the condition may comprise at least one or more of the following situations: The composite component is under force or mechanical load, in particular due to a current dynamic load. The composite component exhibits wear. The composite component exhibits defects in a material of the composite component, the composite component has a load history and/or exhibits aging of a material of the composite component. It is to be understood that other situations are conceivable.

In an advantageous embodiment, the device comprises a measuring device, in particular in the form of a charge amplifier, a voltage amplifier and/or a current amplifier, for increasing a measurement signal, in particular a voltage amplitude of the electrical voltage. By means of a measuring device, in particular in the form of a charge amplifier, the electrical charge separation or an incurred electrical voltage can be improved and determined more precisely. In particular, a measuring amplifier enables a more detailed analysis of individual properties of the electrical charge separation. Furthermore, if a larger amplitude is sampled, a higher resolution of the sampled signal can be achieved when detecting the voltage amplitude. A combination of the aforementioned measuring devices can be achieved by means of a defined closing-off impedance.

The input interface is particularly preferred designed to receive sensor data with information on a mechanical excitation of the composite component, with the analysis unit being designed and configured to determine the condition of the composite component based on the sensor data. In particular, the sensor can comprise a sensor that determines a mechanical excitation of the composite component and generates corresponding sensor data. This way, the electrical charge separation and/or electrical voltage generated can be correlated to the mechanical excitation. A detailed analysis can be performed. In particular, a mechanical excitation already exerting on the composite component can be used to determine the properties of the composite component.

Preferably, the analysis unit is designed and configured to quantify a microphony generated by a mechanical excitation of the composite component and to determine, based on the quantified microphony, a condition, preferably comprising a wear, defects in a material, a load history and/or an aging of a material of the composite component. Hereby, the device can advantageously be used to create a measuring apparatus in which no additional sensors are required to determine a condition of a composite component, in particular directly after production.

In other words, the composite component itself acts as a sensor and, when mechanically excited, generates a corresponding electrical charge separation that provides information on the aforementioned properties of the composite component.

It is to be understood that through the device a mobile measuring station, which is connected to a composite component, can also be created. Then, the composite component can be mechanically excited, for example by an impact or by repetitive oscillations, in particular in the form of vibrations, sound waves or the like. Consequently, a condition of a composite component, such as a bridge, can be effectively determined in a short time period. In particular, it is conceivable to check a bridge for wear at recurring intervals. Thereby, a device as described above is conductively connected to the bridge in predefined time intervals, whereby an excitation of the bridge takes place by driving over the bridge with a motor vehicle. In this case, measurement is possible without blocking the bridge.

It is to be understood that a predefined excitation can also take place. In addition, the excitation of the bridge can be captured by another sensor so that the mechanical excitation of the bridge can be quantified. Even though the example of a bridge has been used here to explain the functioning of the device, this is in no way to be understood as restrictive. A person skilled in the art will recognize that a variety of composite materials and/or composite components can be measured by means of the device.

In a further advantageous embodiment, the analysis unit is designed and configured to quantify a microphony generated by a predefined mechanical excitation of the composite component and, based on the quantified microphony, to determine a composite-component-specific characteristic curve and/or a composite-component-specific characteristic diagram. Similarly, the method preferably includes the step of: excerting on the composite component with a predefined force to generate a predefined mechanical excitation of the composite component; quantifying a microphony generated by the predefined mechanical excitation of the composite component; determining a composite-component-specific characteristic curve and/or a composite-component-specific characteristic diagram. Hereby, preferably under laboratory conditions, characteristic diagrams for different composite materials, such as cables, can be generated, whereby in knowledge of these material-specific characteristic diagrams and/or characteristic curves, a material condition of an already installed cable can be concluded. Consequently, the device enables a person skilled in the art to create a wide-ranging database relating to various composite materials and to use this database to draw conclusions on a wear and/or condition of a composite component during a first measurement. For this purpose, the output interface is preferably designed and configured to transmit the composite component-specific characteristic curve and/or the composite component-specific characteristic diagram to a data memory. Analogously, the following step is preferably provided: transmitting the composite-component-specific characteristic curve and/or the composite-component-specific characteristic diagram to a data memory. It is to be understood that the term “data memory” is to be interpreted broadly. In particular, a data memory may comprise a database, a cloud memory or a removable memory. In particular, it is conceivable that a measuring device in the form of a device as described above is created for measuring a specific cable type, wherein the composite component-specific characteristic diagram and/or the composite component-specific characteristic curve is stored in a memory of the device.

Particularly preferred, a sensor is provided for capturing a mechanical excitation of the composite component and for generating sensor data with information on the mechanical excitation of the composite component, wherein the device is designed and configured to determine the condition of the composite component based on the sensor data. Thereby, a system can be created, that preferably autonomously determines a condition of the composite component upon any mechanical excitation of the composite component. In other words, a monitoring system can be created that determines a condition of the composite component upon any mechanical excitation of the composite component. It is to be understood that such a system may in particular be designed to determine a wear of the composite component and to transmit a corresponding message to a maintenance unit, if the determined wear of the composite component is close to a predefined permissible threshold value for a wear of the composite component.

Advantageously, a method comprises the step of: receiving sensor data including information on a mechanical excitation of the composite component, wherein determining a condition of the composite component comprises determining the condition of the composite component based on the sensor data. By receiving sensor data a mechanical excitation of the composite component can precisely be determined. Determining a condition of the composite component based on the sensor data enables a highly precise analysis of the current condition of the composite component.

Particularly preferred, the step of determining a condition of the composite component comprises determining an exerting mechanical load, preferably based on a composite component-specific characteristic curve and/or a composite component-specific characteristic diagram. Hereby, a mechanical load can be quantified based on the characteristic diagram and/or the characteristic curve. It is to be understood that quantification without a characteristic curve or characteristic diagram is also possible. For bridges in particular, traffic monitoring can be carried out here, for example, since a mechanical load exhibited when a truck drives over the bridge differs greatly from the mechanical load when a bicycle drives over it. Considering a characteristic curve and/or a characteristic diagram, enables a more precise determination of a mechanical excitation.

Advantageously, the step of determining a condition of the composite component comprises determining a wear, a defect in a material, a load history and/or an aging of a material of the composite component, based on any mechanical excitation, preferably based on a composite component-specific characteristic curve and/or a composite component-specific characteristic diagram. This allows the composite component to be monitored for the aforementioned conditions during operation, and in particular allows to monitor wear of the composite component during operation. By means of a component-specific characteristic curve or a component-specific characteristic diagram, the wear of the composite component can be determined with high precision. In particular, this can ensure that composite components are only replaced when they have reached the wear threshold. Preventive replacement of composite components can preferably be omitted.

The presented teaching allows for determining the condition of material and composite conditions of material composites via the detection of mechano-electrical signals or microphony. The newly developed measurement setup can be retrofitted to existing equipment and connected to the material composites to be examined. It is to be understood, that the measurement can be carried out continuously in the scope of quality control after production or as part of preventive maintenance measures with predefined time intervals. Microphony refers in particular to all mechano-electrical effects.

The failure of complex structures or composite materials due to production, load and age can be prevented by this measurement setup and a functional limitation can be detected in time.

Preferably, the newly developed sensor or measuring principle is intended to capture the dynamic wear or defects in vulnerable or relevant mechanical structures, components and components in motion. It is to be understood, that in case of critical wear levels or defects, a hardware connected to the device or the device itself can issue a warning and indicate the wear level or a remaining service life.

In this respect, the term “determining a condition” is to be understood as the determination of a remaining service life.

In particular, the present teachings are based on the knowledge that mechanical loads on conductive and non-conductive material composites generate electrical charge transfers. The magnitude, the temporal progression or the characteristics of the thus generated electrical charge separation is related to the experienced load of the composite materials. These electrical charges and/or electrical voltages can be reliably measured and allow to draw direct conclusions about the condition of the material composites.

It is to be understood that further effects, such as a temperature, in particular a component temperature, preferably caused by an ambient temperature and/or solar radiation; an air flow, in particular a surface wind speed of an air movement at wind turbines; and/or humidity, in particular an air humidity and thus also indirectly a component humidity; can have an influence on the measurement data. Consequently, the aforementioned conditions can be determined based on their influence on the measurement data and the relation of the condition of the composite material to the generated electrical charge separation and/or electrical voltage, and/or the influence on the measurement data can be taken into account in the analysis of the measurement data.

It is further to be understood that the other effects, such as a temperature, an air flow, and/or a humidity, can be determined by external sensors.

Preferably, a correlation of the exerting mechanical load with the measured electrical charge allows a specific analysis of the resulting microphony and its quantification. In a measuring apparatus developed for this purpose, characteristic curves and/or characteristic diagrams for cables and similar material composites can consequently be produced and introduced into the evaluation electronics as a reference for the application.

External loads, i.e. mechanical excitation, can be coupled in via electro-, magneto-, opto- or thermo-mechanical effects or directly mechanically. It goes without saying that in this respect autonomous applications, in which effects already present in a system are used as signal sources and correlated, are also possible.

The evaluated signal allows conclusions to be drawn about the accumulated load history inflicted on the sensor, i.e. the composite material. Thus, both the current load condition and a load history lying in the past can be determined.

With the present teaching, mechano-electric effects in or on a test object to be tested are used to assess a condition of the test object. Here, changes in one or both materials of the composite material can be detected that have a direct or indirect effect on the mechano-electrical effects.

Particularly preferred, verification of defects or changes in composite materials can be created in a new test category.

Quantification, in particular, refers to the specification of properties, qualities and/or characteristics as a numerical value. The term quantification can also include quantization, i.e. digitization of an analog measured value.

In particular, the teaching disclosed herein provides at least one of the following advantages. Timely, non-preventive, replacement of components, structures, media hoses, etc. can be performed. A detection of mechanical wear mechanisms is possible. The device can easily be installed. Preferably, continuous monitoring can be performed by using movements that anyway occur as a reference. A detection of defects directly after production is possible. It is possible to detect load peaks, which in particular can lead to internal damage of a component. Consequential damage and equipment failures can thus be avoided.

A person skilled in the art will recognize that the disclosed teaching can be advantageously used in a variety of applications, including wind turbines, buildings, bridges, and/or vehicle construction. In other words, for both static and dynamic systems.

An embodiment of the invention is described below with reference to the accompanying figures. It shows:

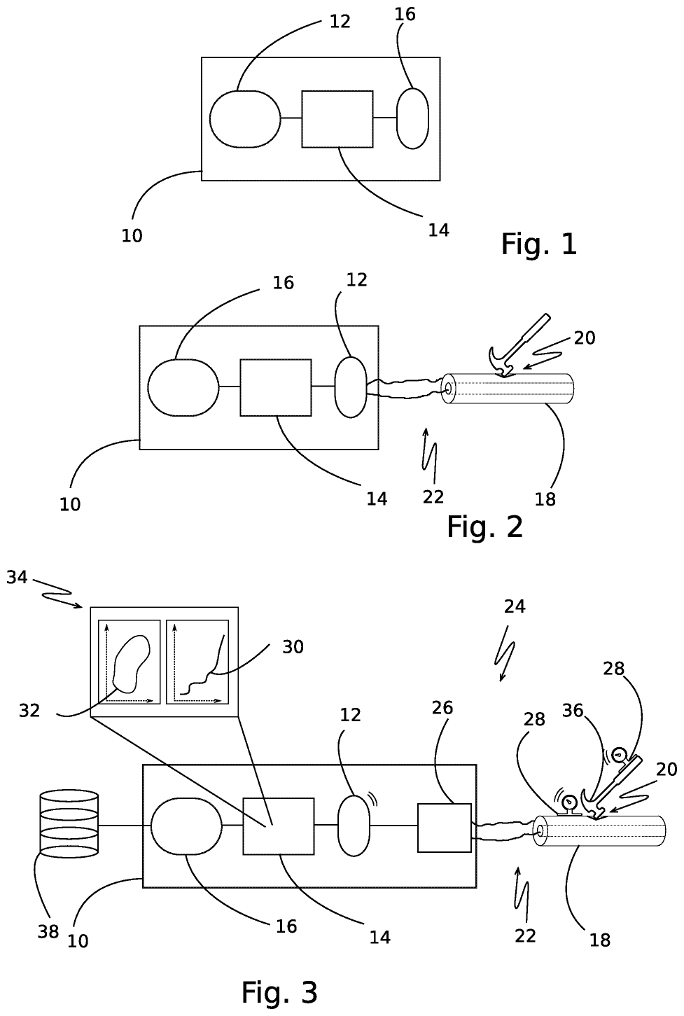

FIG. 1 a schematically simplified view of a device for determining a condition of a composite component;

FIG. 2 a schematically simplified view of a device and a composite component;

FIG. 3 a schematically simplified view of a system for determining a condition of a composite component;

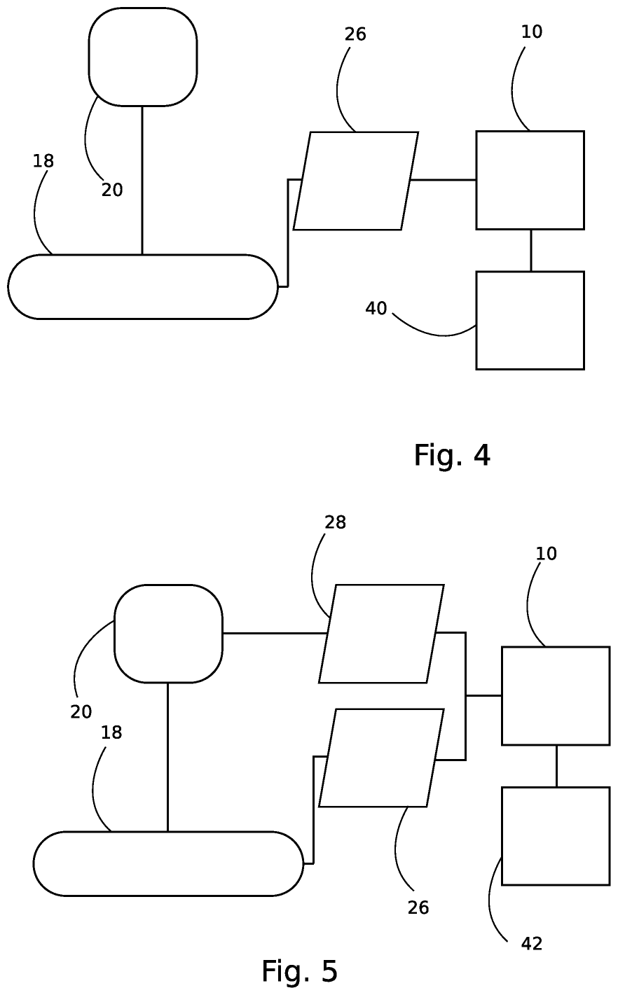

FIG. 4 schematic view of a flow diagram for determining a condition of a composite component;

FIG. 5 schematic view of another flow diagram for determining a condition of a composite component;

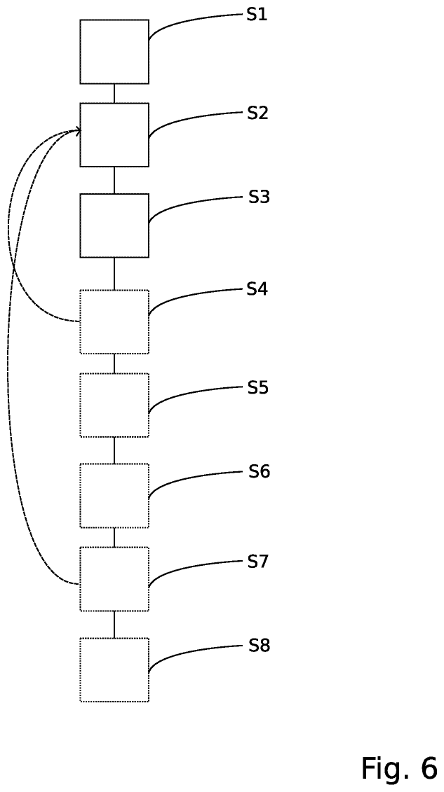

FIG. 6 schematic view of the steps of a method according to the invention; and

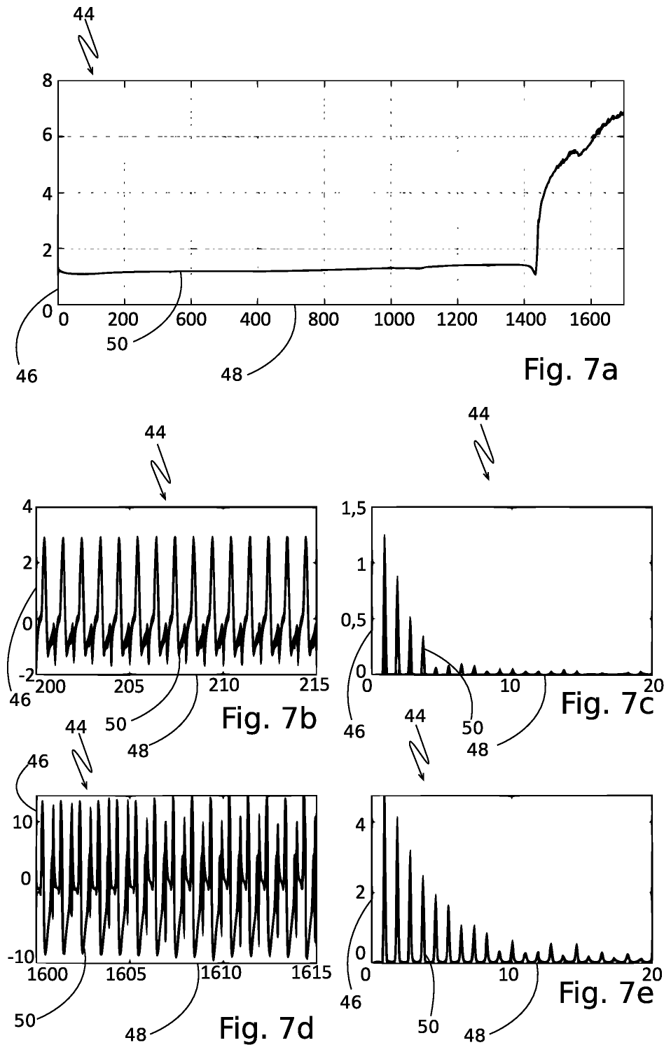

FIG. 7a-7e schematic diagrams of a bending test.

FIG. 1 shows a schematically simplified view of a device 10 for determining a condition of a composite component.

The device 10 includes an input interface 12, an analysis unit 14, and an output interface 16.

The input interface 12 is designed to receive measurement data including information on a condition of the composite component. In particular, the measurement data includes an electrical charge transfer, an electrical charge separation, and/or an electrical voltage generated by a mechanical excitation of the composite component.

The analysis unit 14 determines the condition of the composite component on the basis of the electrical charge transfer, electrical charge separation and/or electrical voltage. For this purpose, the analysis unit makes use of various known analysis methods. It is to be understood that the analysis unit is designed and configured to process, for example, a level and/or decay time, a progression of the electrical charge separation over time, a pulse width, a spectrum, an effective value or the like, for example by means of learning algorithms or machine learning, and to determine the condition, that is, in particular the level of the application of force, a wear, defects in a material and/or a load history or aging of a material of the composite component on the basis of the determined parameters.

FIG. 2 illustrates a device 10 with a composite component 18. In the example shown, the composite component 18 includes a cable having a shield and a core. For clarity reasons, a jacket of the cable is not shown. It is to be understood that in particular the interaction between conductor and insulator, e.g. jacket, may be decisive for the triboelectric effect and only the insulator may be decisive for the piezoelectric effect, and the chosen illustration serves for a better overview.

Schematically shown is a mechanical excitation 20 in the form of a force applied to the composite component 18. To detect an electrical charge transfer and/or electrical voltage, the input interface 12 is connected to the shielding and the core of the composite component 18 via two cables 22. It is to be understood that also only one cable may be provided to determine the electrical charge transfer and/or electrical voltage. It is further to be understood that the composite component 18 may also comprise only one material and a cable may be provided, for example, on the exterior of the composite component 18 to create a composite component 18 from the single-material component, i.e., a component made of only one material. Alternatively, a conductor can be integrated in the composite material and another conductor may be attached to the exterior.

As a result of the force application shown schematically, an electrical charge transfer and/or electrical voltage, in particular a microphony, is generated in the composite component 18, which can be transmitted through the input interface 12 to the analysis unit 14. The analysis unit 14 then determines a condition of the composite component 18, as previously described.

FIG. 3 shows a schematic view of a system 24 for determining a condition of the composite component 18.

In the embodiment shown, the device 10 comprises a measuring unit, in particular a charge amplifier 26.

The charge amplifier 26 is designed to increase an electric charge transfer and/or electric voltage, in particular to increase an amplitude, so that the analysis unit 14 can perform a more detailed analysis. In particular, effects due to the detection of the electric charge transfer and/or electric voltage can be reduced in this case and a signal-to-noise ratio of the desired signal can be increased.

The system 24 further includes optional sensors 28 that can capture the mechanical excitation 20. In particular, the sensors 28 can determine a strength of the mechanical excitation. For this purpose, the sensors 28 communicate, wirelessly in the example shown, with the input interface 12. It is to be understood that only one of the two optional sensors 28 may also be used.

The known characteristics of the mechanical excitation 20, which occurs, for example, during operation of the composite component 18, enable the analysis unit 14 to determine a characteristic curve 30 and/or a characteristic diagram 32 for the composite component 18. The characteristic curve 30 and the characteristic diagram 32 are shown schematically in a graph 34.

For determining the characteristic curve 30 and/or the characteristic diagram 32, an excitation unit 36 can also be applied, which is designed to generate a predefined mechanical excitation 20 in the composite component 18. Here, the predefined mechanical excitation 20 may comprise a single point excitation, a two-dimensional excitation or a time-varying excitation, in particular a vibration or the like.

The output interface 16 is connected to an optional data memory 38 to store the determined characteristic curve 30 or the determined characteristic diagram 32 in the data memory 38.

It is to be understood that it is also conceivable that the data memory 38 communicates with and is connected to the input interface 12, and the analysis unit 14 is designed to read a characteristic curve 30 or a characteristic diagram 32 from the data memory 38 and to determine a condition of the composite component based on the characteristic curve 30 or the characteristic diagram 32.

FIG. 4 shows a schematic view of a flow chart for determining loads in a composite component.

A mechanical excitation 20, in particular an external load, exerts on the composite component 18, whereby an electrical charge transfer, electrical voltage, and/or microphony created is amplified in the measuring device and received and analyzed by the device 10. Preferably, a mechanical load 40 on the composite component 18 may be output by means of the output interface 16 of the device.

FIG. 5 shows a schematic view of a flowchart for predicting a remaining service life of a composite component 18.

In this context, a mechanical excitation 20 takes place in the form of external effects, such as movements and/or vibrations, which exert on the composite component 18 and are additionally captured by a sensor 28. It is to be understood that the sensor 28 is selected based on the effect and may preferably comprise an acceleration sensor. The electrical charge transfer, electrical voltage and/or microphony generated in the composite component 18 is amplified in a measuring device, as already described with respect to FIG. 4, wherein the device 10 receives sensor data from the sensor 28 as well as measurement data from the measuring device and processes them in the courses of a data processing. In particular, a correlation of external effects, i.e. the mechanical excitation 20, and the electrical charge measurement, i.e. the signal of the measuring device or measuring amplifier, can take place in the analysis unit of the device 10. This way, a remaining service life 42 can be predicted.

FIG. 6 shows a schematic view of the steps of a method according to the invention.

In a first step S1, a receiving of measurement data with information on a condition of the composite component takes place.

In a second step S2, a condition of the composite component is determined based on the measurement data.

Finally, in a third step S3, the determined condition of the composite component is transmitted.

The measurement data includes an electrical charge separation and/or an electrical voltage, particularly preferably a microphony, which can be or has been generated by a mechanical excitation of the composite component.

Determining the condition of the composite component includes determining the condition of the composite component based on electrical charge separation, electrical voltage, and/or preferably a microphony.

In an optional fourth step S4, sensor data with information on a mechanical excitation of the composite component is received. In this context, determining of a condition of the composite component, i.e. the second step S2, may comprise determining the condition of the composite component based on the sensor data and the measurement data. In FIG. 6, this is illustrated by a dashed arrow.

In a further or alternative optional fifth step S5, the composite component may be exerted upon with a predefined force to generate a predefined mechanical excitation of the composite component.

The optional fifth step S5 is followed by an optional sixth step S6, which comprises quantifying a microphony generated by the predefined mechanical excitation of the composite component.

In an optional seventh step S7, a composite component-specific characteristic curve and/or a composite component-specific characteristic diagram is determined.

In a particularly preferred optional eighth step S8, the composite component-specific characteristic curve and/or the composite component-specific characteristic diagram is transmitted to a data memory. It is to be understood that the determined characteristic diagram or the determined characteristic curve can also be taken into account in the second step S2 of determining a condition of the composite component. In particular, existing knowledge from a database can be contributed when determining the condition. It is to be understood that the data analysis can be based on machine learning.

Consequently, the second step S2 of determining a condition of the composite component may preferably comprise determining an exerting mechanical load, preferably based on a composite component-specific characteristic curve and/or a composite component-specific characteristic diagram.

In another preferred embodiment, the second step S2 of determining a condition of the composite component may comprise determining a wear, a defect in a material, a load history and/or an aging of a material of the composite component based on any mechanical excitation, preferably based on a composite component-specific characteristic curve and/or a composite component-specific characteristic diagram.

FIG. 7a shows an effective value of an electric charge transfer in a bending test plotted against the number of cycles.

In this bending test, a coaxial cable, in particular a coaxial cable of type CLF200, was examined in accordance with the bending test according to DIN EN50396.

The coaxial cable has a rigid copper conductor with a 1 mm² cross-section as the inner conductor, which is insulated from a shielding by a dielectric in the form of polyethylene (PE). The shielding includes an aluminum foil and a copper braiding. This coaxial cable has a jacket made of PVC.

In the bending test, the cable is loaded with a bending radius of 50 mm, at an angle of ±90° and with 60 repetitions per minute. Break detection is performed by means of current monitoring, preferably at 0.1 A, with the inner and outer conductors connected in series.

FIG. 7a shows a graph 44 of the effective value of the electrical charge transfer over a number of cycles or repetitions.

On the y-axis the effective value, for example the root mean square, RMS, of the electric charge transfer is plotted in root of picocoulomb square \sqrt{pC²}. On the x-axis the repetitions are plotted.

The y-axis 46 ranges from 0 to 8 in the graph 44 shown in FIG. 7a. The x-axis 48 ranges from 0 to over 1600 in the graph shown in FIG. 7a, with an x-axis tic marking each 200 cycles.

A curve 50 of the effective value of the electric charge exhibits an approximately flat progression in a range between 0 and about 1,400 repetitions, with a value for the effective electric charge transfer of about 1 root picocoulomb square \sqrt{pC²}. Thereafter, the curve 50 falls slightly and rises sharply to a value of more than 6 root picocoulomb square \sqrt{pC²} at over 1,600 repetitions.

FIG. 7b shows a graph 44 of a temporal progression of the repetitions 200 to 215, where the y-axis 46 covers a range from −2 to 4 picocoulomb pC.

FIG. 7c illustrates the temporal progression shown in FIG. 7b within the frequency domain, where the x-axis 48 covers a range from 0 to 20 Hz and the y-axis 46 covers a range from 0 to 1.5 absolute value of picocoulomb.

In a manner analogous to FIGS. 7b and 7c, FIGS. 7d and 7e show a progression in the time domain and in the frequency domain.

Unlike graph 44 shown in FIG. 7b, the y-axis 46 in FIG. 7d ranges from −10 to over 10 picocoulomb pC, where the repetitions 1600 to 1615 are shown.

In FIG. 7e, the y-axis 46 ranges from 0 to above 4 absolute value picocoloumb |pC|.

When comparing FIGS. 7b, 7c with FIGS. 7d, 7e, it becomes apparent that the electric charge transfer has a higher amplitude. Furthermore, it can be seen that in the frequency domain, the electric charge transfer has more components in the higher frequency range, especially between 10 and 20 Hz. In addition, frequency components greater than 5 Hz are also more pronounced.

The invention has been comprehensively described and explained with reference to the drawings and description. The description and explanation are intended to be exemplary and not limiting. The invention is not limited to the disclosed embodiments. Other embodiments or variations will become apparent to those skilled in the art upon use of the present invention and upon close analysis of the drawings, the disclosure and the following claims.

In the claims, the words “comprising” and “with” do not exclude other elements or steps. The indefinite article “a” or “an” does not exclude a plurality. A single element or unit may fulfill the functions of several items recited in the claims. An element, a unit, a device, and a system may partially or completely be implemented by corresponding hardware and/or software. The mere fact that certain measures are recited in mutually different dependent claims does not indicate that a combination of these measures cannot be used to advantage. A computer program may be stored/distributed on a non-volatile medium, for example, on an optical memory or on a solid state drive (SSD). A computer program may be distributed in combination with hardware and/or as part of a hardware, for example, by means of the Internet or by means of wired or wireless communication systems. Reference signs in the patent claims are not to be understood restrictively.

### REFERENCE SIGNS

10 Device

12 Input interface

14 Analysis unit

16 Output interface

18 Composite component

20 Mechanical excitation

22 Cable

24 System

26 Charge amplifier

28 Sensor

30 Characteristic curve

32 Characteristic diagram

34 Graph

36 Excitation unit

38 Data storage

40 Load

42 Remaining service life

44 Graph

46 Y-axis

48 X-axis

50 Curve

S1-S3 Method steps

S4-S8 Optional method steps

1-12. (canceled)

13. A device for determining a condition of a composite component, comprising:
an input interface for receiving measurement data with information on a condition of the composite component;
an analysis unit for determining a condition of the composite component based on the measurement data; and
an output interface for transmitting the determined condition;
wherein
the measurement data comprises an electrical charge transfer and/or electrical voltage, which can be generated by the composite component through mechanical excitation of the composite component; and
the analysis unit is designed and configured to determine the condition of the composite component on the basis of the electrical charge separation and/or electrical voltage.

14. The device according to claim 13, having a measuring device for increasing a measurement signal.

15. The device according to claim 13, having a charge amplifier or a voltage amplifier for increasing a voltage amplitude of the electrical voltage.

16. The device according to claim 13, wherein the input interface is designed to receive sensor data including information on a mechanical excitation of the composite component; and the analysis unit is designed and configured to determine the condition of the composite component based on the sensor data.

17. The device according to claim 13, wherein the analysis unit is designed and configured to quantify a microphony generated by a mechanical excitation of the composite component, and based on the quantified microphony, to determine the condition of the composite component.

18. The device according to claim 13, wherein the condition comprises a wear, defects in a material, a load history and/or an aging of a material, of the composite component.

19. The device according to claim 13, wherein the analysis unit is designed and configured to quantify a microphony generated by a predefined mechanical excitation of the composite component, and to determine a composite component-specific characteristic curve and/or a composite component-specific characteristic diagram based on the quantified microphony.

20. The device according to claim 13, wherein the analysis unit is designed and configured to quantify a microphony generated by a predefined mechanical excitation of the composite component, and to determine a composite component-specific characteristic curve and/or a composite component-specific characteristic diagram based on the quantified microphony and wherein the output interface is designed and configured to transmit the composite-component-specific characteristic curve and/or the composite-component-specific characteristic diagram to a data memory.

21. A system for determining a condition of a composite component, comprising:
a device according to claim 13; and
an excitation unit for exerting on the composite component and generating a predefined mechanical excitation of the composite component.

22. The system according to claim 21, comprising:
a sensor for capturing a mechanical excitation of the composite component and generating sensor data including information on the mechanical excitation of the composite component; wherein
the device is designed and configured to determine the condition of the composite component additionally based on the sensor data.

23. A method of determining a condition of a composite component comprising the steps of:
receiving measurement data with information on a condition of the composite component;
determining a condition of the composite component based on the measurement data; and
transmitting the determined condition;
wherein
the measurement data comprises an electrical charge separation and/or electrical voltage generated by the composite component through mechanical excitation of the composite component; and
determining a condition of the composite component comprises determining the condition of the composite component based on the electrical charge separation and/or electrical voltage.

24. The method according to claim 23, comprising the step of:
receiving sensor data with information on a mechanical excitation of the composite component;
wherein determining a condition of the composite component comprises determining the condition of the composite component additionally based on the sensor data.

25. The method according to claim 23, comprising the steps of:
exerting on the composite component with a predefined force to generate a predefined mechanical excitation of the composite component;
quantifying a microphony generated by the predefined mechanical excitation of the composite component;
determining a composite component-specific characteristic curve and/or a composite component-specific characteristic diagram.

26. The method according to claim 23, wherein the step of determining a condition of the composite component comprises determining an exerting mechanical load.

27. The method according to claim 23, wherein the step of determining a condition of the composite component comprises determining an exerting mechanical load based on a composite component-specific characteristic curve and/or a composite component-specific characteristic diagram.

28. The method according to claim 23, wherein the step of determining a condition of the composite component comprises determining a wear, a defect in a material, a load history and/or an aging of a material of the composite component based on any mechanical excitation.

29. The method according to claim 23, wherein the step of determining a condition of the composite component comprises determining a wear, a defect in a material, a load history and/or an aging of a material of the composite component additionally based on a composite component-specific characteristic curve and/or one or more composite component-specific characteristic diagrams.
